(19) United States
(12) Patent Application Publication
(10) Pub. No.: US 2026/0151397 A1
(43) Pub. Date: Jun. 4, 2026

(54) PHARMACOLOGICAL INHIBITION OF MEK CAN IMPROVE OUTCOMES OF HYPERTROPHIC CARDIOMYOPATHY (HCM) IN INDIVIDUALS WITH NOONAN SYNDROME WITH MULTIPLE LENTIGINES (NSML)

(71) Applicant: Northwestern University, Evanston, IL (US)

(72) Inventors: Carlos E. PRADA, Chicago, IL (US); Anna Gah-Mei JOONG, Chicago, IL (US)

(73) Assignee: Northwestern University, Evanston, IL (US)

(21) Appl. No.: 19/407,664

(22) Filed: Dec. 3, 2025

Related U.S. Application Data
(60) Provisional application No. 63/727,934, filed on Dec. 4, 2024.

(51) Int. Cl. A61K 31/519; A61P 9/04; G01N 33/68

(52) U.S. Cl. CPC A61K 31/519; A61P 9/04; G01N 33/6893; G01N 2333/58; G01N 2800/325; G01N 2800/52

(57) ABSTRACT

Provided are methods of treating hypertrophic cardiomyopathy patients having Noonan Syndrome with multiple lentigines, the method including treating the patient with an MEK inhibitor.

### CROSS-REFERENCE TO RELATED APPLICATIONS

This application claims the benefit of priority to U.S. Provisional Patent Application No. 63/727,934, filed on Dec. 4, 2024, the contents of which are incorporated herein by reference in their entirety.

### BACKGROUND

Hypertrophic cardiomyopathy (HCM) develops in close to 90% of individuals with Noonan syndrome with multiple lentigines (NSML). HCM is progressive with high morbidity and mortality in the NSML population. NSML is caused by specific variants in the PTPN11 gene that causes a dominant negative catalytically defective protein leading to enhanced activation of the PI3K-AKT-mTOR pathway. This is a significantly different mechanism for HCM in Noonan syndrome PTPN11 pathogenic variants which lead to gain of function alleles and increased RAS-MAPK pathway activation. Therapy for HCM in NSML is limited to symptomatic management and use of beta-blockers and there is often need for myectomy, implantable cardioverter defibrillator-shock, and heart transplantation. Experimental data using mTOR inhibitors in mice models and a single patient treated with rapamycin showed limited benefit with ultimate need for heart transplantation.

There is a need in the art for therapy for HCM type in NSML, and the present disclosure satisfied this need.

### SUMMARY

According to an aspect of the present technology, a method for treating HCM in a subject having NSML is provided, the method comprising administering to the subject an MEK inhibitor.

In an aspect, provided is a method of treating hypertrophic cardiomyopathy (HCM) in a subject in need thereof, the method comprising administering to the subject an effective amount of a MEK inhibitor.

In some embodiments, the subject is diagnosed with Noonan syndrome with multiple lentigines (NSML).

In some embodiments, the MEK inhibitor comprises trametinib.

In some embodiments, the effective amount of the MEK inhibitor is from about 0.012 mg/kg/day to about 0.025 mg/kg/day.

In some embodiments, the MEK inhibitor is administered orally.

In some embodiments, the MEK inhibitor is administered for a period of time from about 1 week to about 30 months.

In another aspect, provided is a method of treating HCM in a subject diagnosed with NSML, the method comprising administering to the subject an effective amount of trametinib.

In some embodiments, the effective amount of trametinib is from about 0.012 mg/kg/day to about 0.025 mg/kg/day.

In some embodiments, trametinib is administered orally.

In some embodiments, the trametinib is administered for a period of time from about 1 week to about 30 months.

In yet another aspect, provided is a method for determining response to therapy in a subject having NSML related HCM, the method comprising: measuring an initial level of NT-pro-BNP in the subject; treating the subject with an MEK inhibitor; and measuring a second level of NT-pro-BNP in the subject.

In some embodiments, when the second level of NT-pro-BNP in the subject is lower than the initial level of NT-pro-BNP, the subject is responding to therapy with the MEK inhibitor.

In some embodiments, the MEK inhibitor is trametinib.

In some embodiments, the method further comprises measuring the level of NT-pro-BNP over a period of time where the patient is treated with the MEK inhibitor.

In some embodiments, the period of time is from 1 week to 30 months.

In some embodiments, the level of NT-pro-BNP is measured daily, weekly, or monthly.

Both the foregoing summary and the following description of the drawings and detailed description are exemplary and explanatory. They are intended to provide further details of the disclosure but are not to be construed as limiting. Other objects, advantages, and novel features will be readily apparent to those skilled in the art from the following detailed description of the disclosure.

### BRIEF DESCRIPTION OF THE DRAWINGS

FIG. 1A shows the different pathways involved in hypertrophic cardiomyopathy (HCM) 1s in normal subjects, and subjects having Noonan Syndrome (NS) and Noonan Syndrome with multiple lentigines (NSML) (top panels), and the effects of treatment with an MEK inhibitor on HCM in the subjects (bottom panels).

FIG. 1B depicts the differences between the heart of a normal subject (top) and the heart of a subject having hypertrophic cardiomyopathy (MCM, bottom). In subjects having MCM, the walls of the left ventricle become thick and stiff.

FIG. 1C shows a schematic demonstrating that MEK inhibition is better than mTOR inhibition for treating MCM in subjects having NSML. Either primary or secondary RAS activation in the highlighted panel is the possible mechanism for why a MEK inhibitor works to treat HCM.

FIG. 2 shows a summary of Patient 1 and Patient 2, both patients having NSML and underwent treatment for HCM with an MEK inhibitor (trametinib).

FIG. 3A shows the results of treatment of Patient 1 with an mTOR inhibitor (sirolimus) for four months, then with an MEK inhibitor (trametinib) for 27 months. N-terminal pro b-type natriuretic peptide (NT-pro-BNP) and interventricular septum thickness (IVSD) were measured over the course of treatment. Patient 1 showed no response to treatment with an mTOR inhibitor, which has been proposed as the signaling pathway that drives hypertrophic cardiomyopathy (HCM) in individuals with NSML. Treatment with trametinib (an MEK inhibitor) showed rapid significant improvement of NT-pro-BNP levels in this individual with severe HCM, and Patient 1 had meaningful clinical outcomes such as extubation, feeding tolerance, and discharge from hospital with survival. There was a biomarker rebound when the dose was adjusted for side effects, followed by a sustained response at a lower dose of MEK inhibitor with improvement of IVSd scores in cardiac imaging.

FIG. 3B shows the results of treatment of Patient 2 with trametinib for over 15 months. NT-pro-BNP and IVSd were measured over the course of treatment. Treatment with trametinib showed rapid significant improvement of NT-pro-BNP in Patient 2, who had HCM. Notably, there is a clear rebound in biomarker levels when the dose was adjusted to manage side effects, followed by a sustained biomarker response with intermittent dosing of the MEK inhibitor. Additionally, as the dose was gradually increased in frequency from intermittent to daily and from 0.010 mg/kg to 0.020 mg/kg without recurrence of side effects, further cardiac remodeling was observed with improvements in IVSD z-score.

FIG. 4 shows the heart failure classification of Patients 1 and 2 pre- and post-therapy with an MEK inhibitor.

### DETAILED DESCRIPTION

### Overview

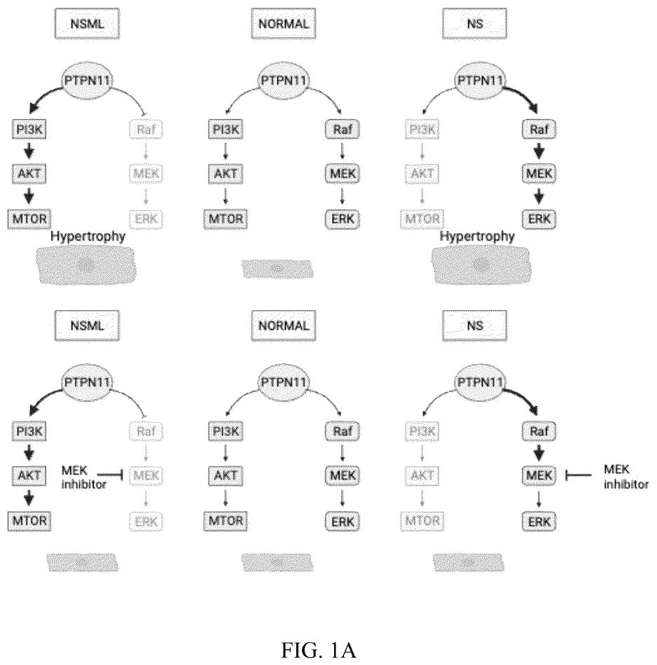

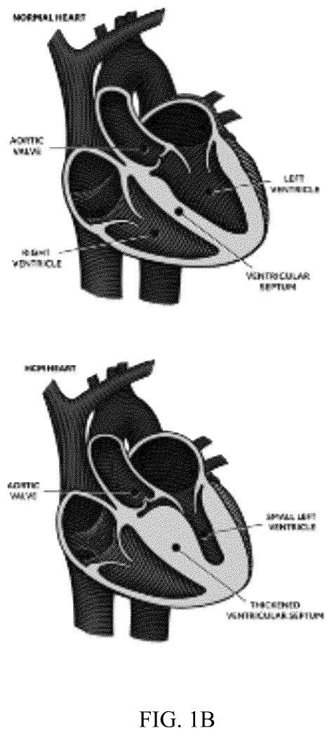

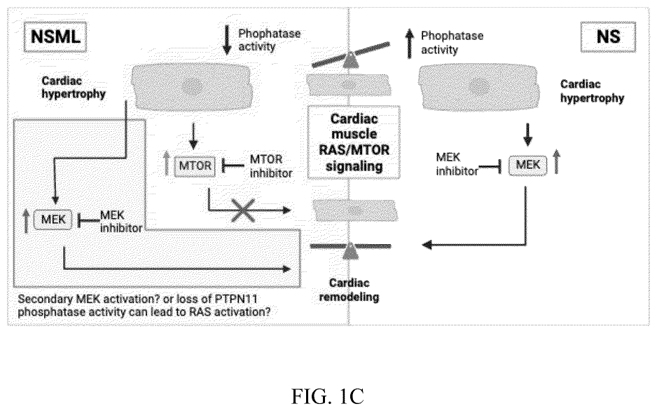

Hypertrophic cardiomyopathy (HCM) develops in close to 90% of individuals with Noonan syndrome with multiple lentigines (NSML). HCM is progressive with high morbidity and mortality in the NSML population, and there are limited interventions at this time. NSML is caused by specific variants in the PTPN11 gene that causes a dominant negative catalytically defective protein leading to enhanced activation of the PI3K-AKT-mTOR pathway. This is a significantly different mechanism for HCM in Noonan syndrome pathogenic variants which lead to gain of function alleles and increased RAS-MAPK pathway activation (FIG. 1A). Therapy for HCM in NSML is limited to symptomatic management and use of beta-blockers and there is often need for myectomy, implantable cardioverter defibrillator-shock, and heart transplantation.

Current pharmacological approaches for treating HCM in NSML have focused on using mTOR inhibitors, such as sirolimus and everolimus. However, experimental data using mTOR inhibitors in mice models and a single patient treated with rapamycin showed limited benefit with ultimate need for heart transplantation.

Provided herein are novel therapeutic approaches for individuals with severe hypertrophic cardiomyopathy (HCM) due to variants associated with Noonan syndrome with multiple lentigines. The methods described herein provide therapy for HCM in patients having NSML using an MEK inhibitor, which is a different pharmacological target to current dogma with clinical and biomarker data supporting improvement of heart failure/HCM. Pharmacological therapy with low dose MEK inhibitors leads to improved clinical outcomes of HCM in individuals with NSML, including extubation, feeding tolerance, and survival.

Provided herein is the identification of a new target for human disease and a biomarker to monitor response to intervention and echocardiogram improvement. NT-pro-BNP is a good biomarker for response and dose titration of MEK inhibitor therapy in NSML related HCM.

### Definitions

The use of the terms “a” and “an” and “the” and similar referents in the context of describing the elements (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

“Administration” of a disclosed compound encompasses the delivery to a subject of a compound as described herein, or a prodrug or other pharmaceutically acceptable derivative thereof, using any suitable formulation or route of administration, as discussed herein.

The term “co-administration,” “administered in combination with,” and their grammatical equivalents, as used herein, encompasses administration of two or more agents to the subject so that both agents and/or their metabolites are present in the subject at the same time. Co-administration includes simultaneous administration in separate compositions, administration at separate times in separate compositions, or administration in a composition in which both agents are present.

As used herein, the terms “treatment”, “treating”, “palliating” “managing” and “ameliorating” are used interchangeably herein. These terms refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying, or eliminating the onset of symptoms of a disease or condition, slowing, halting, or reversing the progression of a disease or condition, or any combination thereof.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

“Pharmaceutically acceptable carrier” or “pharmaceutically acceptable excipient” includes any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. The use of such media and agents for pharmaceutically active substances is well known in the art. Except insofar as any conventional media or agent is incompatible with the active ingredient, its use in the therapeutic compositions as disclosed herein is contemplated. Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as D-alpha-tocopherol polyethylene glycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β, and γ-cyclodextrin, or chemically modified derivatives such as hydroxyalkylcyclodextrins, including 2- and 3-hydroxypropyl-cyclodextrins, or other solubilized derivatives can also be used to enhance delivery of compounds described herein.

### Methods of Treating HCM in Subjects Having NSML

Provided herein are methods for treating HCM in subjects having NSML. The provided methods comprise administering to the subject an effective amount of a MEK inhibitor.

Treatment with low doses of MEK inhibitors leads to improved clinical outcomes of HCM in individuals with NSML, including extubation, feeding tolerance, and survival.

The MEK inhibitor may be trametinib, which has the following structure:

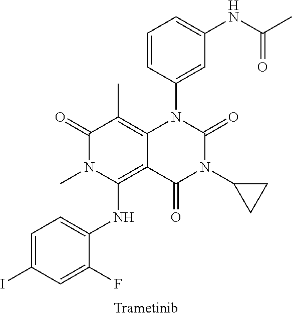 [SMILES: CC(=O)Nc1cccc(-n2c(=O)n(C3CC3)c(=O)c3c(Nc4ccc(I)cc4F)n(C)c(=O)c(C)c32)c1]

Trametinib is approved for treatment of adult cancer with a somatic BRAF pathogenic variant in the MAPK signaling pathway but has not been approved for the treatment of pediatric patients nor non-malignant illnesses such as RASopathies.

The subject may be treated with a low dose of the MEK inhibitor. In some embodiments, the low dose is from about 0.01 mg/kg/day to about 0.03 mg/kg/day of the MEK inhibitor.

In some embodiments, the subject is treated with about 0.012 mg/kg/day to about 0.025 mg/kg/day of trametinib. In some embodiments, the subject is treated with about 0.010 mg/kg/day, 0.011 mg/kg/day, 0.012 mg/kg/day, 0.013 mg/kg/day, 0.014 mg/kg/day, 0.015 mg/kg/day, 0.016 mg/kg/day, 0.017 mg/kg/day, 0.018 mg/kg/day, 0.019 mg/kg/day, 0.020 mg/kg/day, 0.021 mg/kg/day, 0.022 mg/kg/day, 0.023 mg/kg/day, 0.024 mg/kg/day, or 0.025 mg/kg/day of trametinib.

In some embodiments, the MEK inhibitor is administered orally.

In some embodiments, the subject is treated with the MEK inhibitor for a period of time ranging from about 1 week to about 30 months. In some embodiments, the period of time is about 1 week, about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, about 27 months, about 28 months, about 29 months, or about 30 months.

In some embodiments, the patient is a pediatric patient.

Provided herein are methods for determining a subject's response to therapy with an MEK inhibitor. The subject may be diagnosed with NSML related MCM. The method may comprise: measuring an initial level of NT-pro-BNP in the subject; treating the subject with the MEK inhibitor; and measuring a second level of NT-pro-BNP in the subject. Treatment with the MEK inhibitor may be successful when the second level of NT-pro-BNP in the subject is lower than the initial level of NT-pro-BNP in the subject. NT-pro-BNP level may also be used in a dose titration of MEK inhibitor therapy in NSML related HCM.

NT-pro-BNP levels may be measured multiple times during the course of therapy with an MEK inhibitor. As an example, NT-pro-BNP levels may be measured after administration of each dose of MEK inhibitor, NT-pro-BNP levels may be measured weekly, or NT-pro-BNP levels may be measured monthly.

### Methods of Administration

In general, the disclosed methods comprise administering to a subject an MEK inhibitor in an effective amount. An “effective amount” with reference to an MEK inhibitor of the present disclosure means an amount of the MEK inhibitor that is sufficient to engage the target (e.g., by inhibiting the target) at a level that is indicative of the potency of the compound. Target engagement can be determined by one or more biochemical or cellular assays resulting in an EC50, ED50, EC90, IC50, or similar value which can be used as one assessment of the potency of the compound. Assays for determining target engagement include, but are not limited to, those described in the Examples. The effective amount may be administered as a single quantity or as multiple, smaller quantities (e.g., as one tablet with “x” amount, as two tablets each with “x/2” amount, etc.).

In some embodiments, the disclosed methods comprise administering a therapeutically effective amount of an MEK inhibitor to a subject in need thereof. As used herein, the phrase “therapeutically effective amount” with reference to compound disclosed herein means a dose regimen (i.e., amount and interval) of the MEK inhibitor that provides the specific pharmacological effect for which the MEK inhibitor is administered to a subject in need of such treatment. For treatment, a therapeutically effective amount may be effective to reduce, ameliorate, or eliminate one or more signs or symptoms associated with a disease, delay disease progression, prolong survival, decrease the dose of other medication(s) required to treat the disease, or a combination thereof. A therapeutically effective amount may vary based on, for example, one or more of the following: the age and weight of the subject, the subject's overall health, the stage of the subject's disease, the route of administration, and prior or concomitant treatments.

Administration may comprise one or more (e.g., one, two, or three, or more) dosing cycles. Administration may comprise oral administration, parenteral administration, or any administration method suitable for treatment with the MEK inhibitor.

In certain embodiments, the compounds contemplated by the present disclosure may be administered (e.g., orally) at about 0.01 mg/kg to about 0.03 mg/kg of subject's body weight per day, one or more times a day, a week, or a month, to obtain the desired effect. In some embodiments, once daily or twice daily administration is contemplated. In some embodiments, a suitable weight-based dose of a compound contemplated by the present disclosure is used to determine a dose that is administered independent of a subject's body weight. In certain embodiments, the compounds of the present disclosure are administered (e.g., orally, parenterally, etc.) at fixed dosage levels of about 1 mg to about 100 mg, particularly 1, 3, 5, 10, 15, 20, 25, 50, 75, 100 mg, one or more times a day, a week, or a month, to obtain the desired effect.

In certain embodiments, the compound is contained in a “unit dosage form”. The phrase “unit dosage form” refers to physically discrete units, each unit containing a predetermined amount of the compound, either alone or in combination with one or more additional agents, sufficient to produce the desired effect. It will be appreciated that the parameters of a unit dosage form will depend on the particular agent and the effect to be achieved.

### Kits

In one aspect, provided is a kit for use in a method as disclosed herein.

In some embodiments, the kit comprises, or alternatively consists essentially of, or yet further consist of instructions for use and: a peptide as disclosed herein or a pharmaceutical composition as disclosed herein. In further embodiments, the kit is suitable for use in a method of treatment as disclosed herein.

The present technology, thus generally described, will be understood more readily by reference to the following examples, which are provided by way of illustration and are not intended to be limiting of the present technology.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The present invention, thus generally described, will be understood more readily by reference to the following examples, which are provided by way of illustration and are not intended to be limiting of the present invention.

### Examples

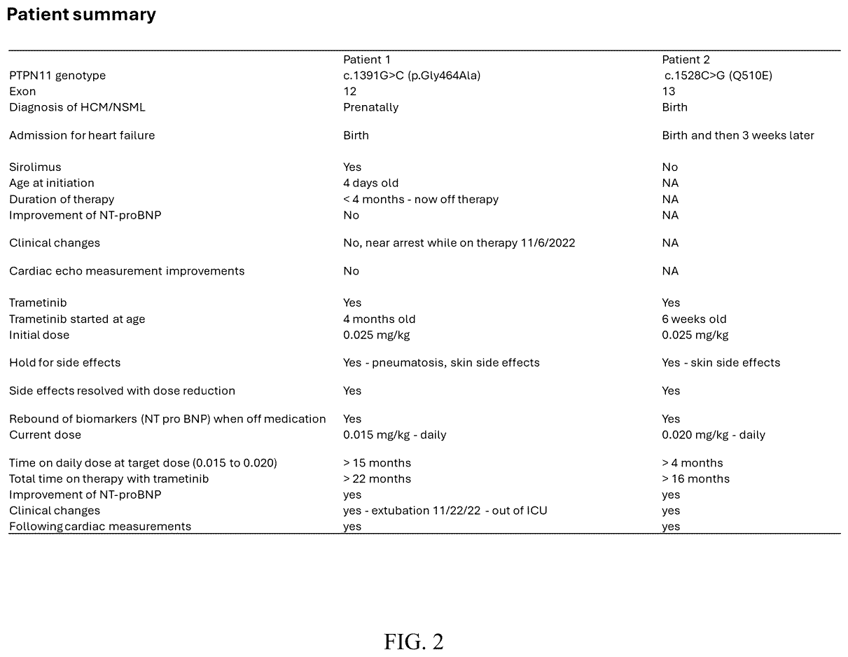

Pharmacological inhibition of MEK can improve outcomes of HCM in individuals with NSML. Provided are clinical and laboratory data and outcomes of therapy with MEK inhibitors in two individuals with unresponsive cardiomyopathy to standard of care. A summary of the patient information and summary of the treatment is provided in FIG. 2.

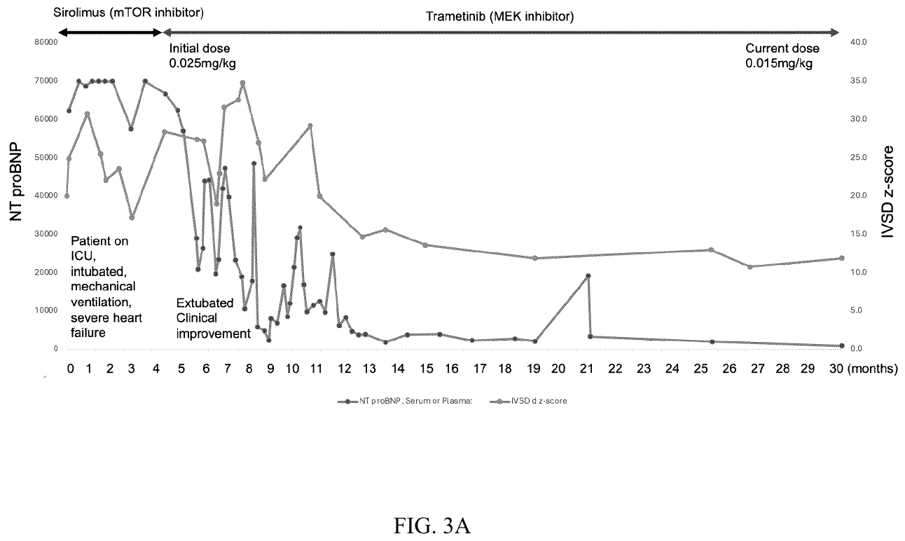

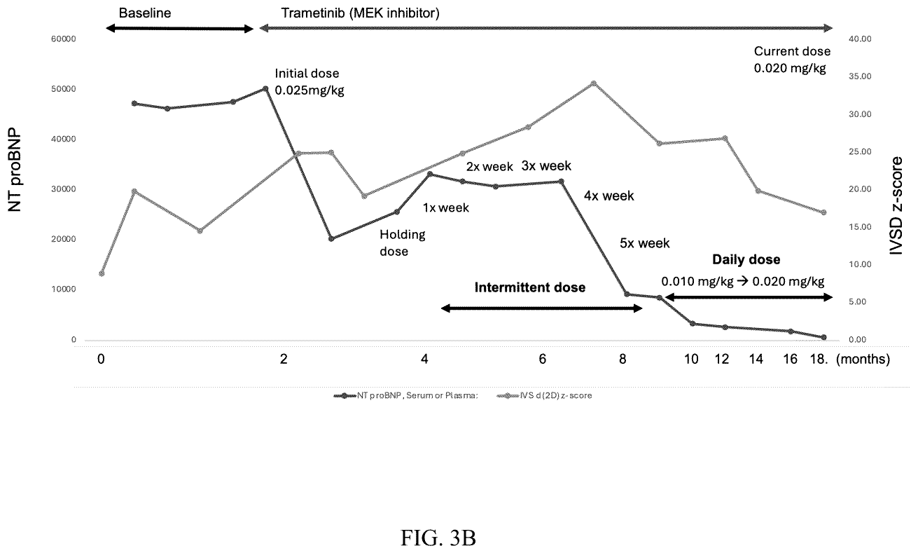

Therapy with an MEK inhibitor (trametinib) successfully showed rapid significant improvement of NT-pro-BNP in two individuals with severe HCM (FIGS. 3A-3B). NT-pro-BNP is a known biomarker of heart failure and is used to monitor the effectiveness of treatment in this population. NT-pro-BNP levels appeared to be MEK inhibitor dose dependent (the initial dose administered to both patients was 0.025 mg/kg/day, and the current dose was 0.015 mg/kg/day for Patient 1 and 0.020 mg/kg/day for Patient 2, with better tolerance of medication and sustained response. There is also a rebound of NT-pro-BNP when MEK inhibitor was temporarily held, showing a direct correlation with intervention (observed in these two individuals). In Patient 1, there was no response (no NT-pro-BNP or clinical improvement) within 4 months of treatment with sirolimus (an mTOR inhibitor), contrary to what is reported in the literature in the few published cases. A week after initiation of MEK inhibition to Patient 1 resulted in a significant reduction of NT-proBNP, and the patient was extubated shortly and was able to initiate feeds. Patient 2 was initiated in therapy with the MEK inhibitor during initial admission and had not been readmitted for cardiac decompensation. Patient 2 showed stable echocardiogram findings with sustained improvement of NT-pro-BNP after 5 months on therapy.

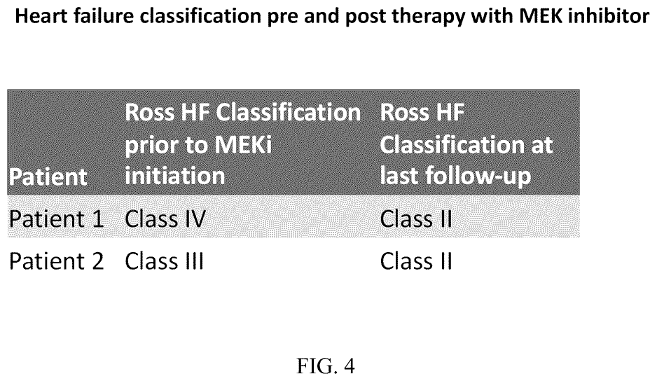

The overall interventricular septum thickness (IVSD) decreased over time in at least one individual (Patient 1, FIG. 3A), who was on therapy for a longer duration (27 months). Patient 1 also showed meaningful clinical outcomes such as extubation, feeding tolerance, and discharge from hospital with survival. The heart failure classification for Patient 1 went from Class IV pre-therapy to Class II post-therapy with trametinib, and the heart failure classification for Patient 2 went from Class III pre-therapy to Class II post-therapy with trametinib (FIG. 4).

The clinical and laboratory data from pharmacological MEK inhibition in these two individuals supports a different approach to the dogma that loss of function variants in PTPN11 with reduced protein catalytic activity will not benefit from MEK inhibitors.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method of treating hypertrophic cardiomyopathy (HCM) in a subject in need thereof, the method comprising administering to the subject an effective amount of a MEK inhibitor.

2. The method of claim 1, wherein the subject is diagnosed with Noonan syndrome with multiple lentigines (NSML).

3. The method of claim 1, wherein the MEK inhibitor comprises trametinib.

4. The method of claim 1, wherein the effective amount is from about 0.012 mg/kg/day to about 0.025 mg/kg/day.

5. The method of claim 1, wherein the MEK inhibitor is administered orally.

6. The method of claim 1, wherein the MEK inhibitor is administered for a period of time from about 1 week to about 30 months.

7. A method of treating HCM in a subject diagnosed with NSML, the method comprising administering to the subject an effective amount of trametinib.

8. The method of claim 7, wherein the effective amount is from about 0.012 mg/kg/day to about 0.025 mg/kg/day.

9. The method of claim 7, wherein trametinib is administered orally.

10. The method of claim 7, wherein trametinib is administered for a period of time from about 1 week to about 30 months.

11. A method for determining response to therapy in a subject having NSML related HCM, the method comprising:
measuring an initial level of NT-pro-BNP in the subject;
treating the subject with an MEK inhibitor; and
measuring a second level of NT-pro-BNP in the subject.

12. The method of claim 11, wherein when the second level of NT-pro-BNP in the subject is lower than the initial level of NT-pro-BNP, the subject is responding to therapy with the MEK inhibitor.

13. The method of claim 11, wherein the MEK inhibitor is trametinib.

14. The method of claim 11, further comprising measuring the level of NT-pro-BNP over a period of time where the patient is treated with the MEK inhibitor.

15. The method of claim 14, wherein the period of time is from 1 week to 30 months.

16. The method of claim 14, wherein the level of NT-pro-BNP is measured daily, weekly, or monthly.
